(19) United States
(12) Patent Application Publication
(10) Pub. No.: US 2008/0027031 A1
(43) Pub. Date: Jan. 31, 2008

(54) Retinoids and Related Compounds for the Treatment of Neuroinflammatory Conditions, Diseases and Disorders

(75) Inventors: Marc K. Hellerstein, Kensington, CA (US); Michael Marino, Clifton Park, NY (US); Mahalakshmi Shankaran, Walnut Creek, CA (US)

Correspondence Address: MORGAN, LEWIS & BOCKIUS, LLP, ONE MARKET SPEAR STREET TOWER, SAN FRANCISCO, CA, 94105, US

(73) Assignee: KineMed, Inc., Emeryville, CA (US)

(21) Appl. No.: 11/683,931

(22) Filed: Mar. 8, 2007

Related U.S. Application Data
(60) Provisional application No. 60/780,793, filed on Mar. 8, 2006.
(60) Provisional application No. 60/780,749, filed on Mar. 8, 2006.

(51) Int. Cl. A61K 31/65; A61K 31/07; A61P 29/00; A61K 31/56

(52) U.S. Cl. 514/154; 514/171; 514/725

(57) ABSTRACT

The proliferation and activation of microglia is emerging as an important etiologic factor and target for therapeutic intervention in several CNS diseases, including Alzheimer's disease and multiple sclerosis. Here, we have discovered that retinoic acids dramatically inhibit microglial proliferation in an in vivo model including the widely-used animal model of multiple sclerosis, the experimental autoimmune encephalomyelitis (EAE) mouse model.

### CROSS REFERENCE TO RELATED APPLICATIONS

This application claims the benefit under 35 U.S.C. §119(e) of U.S. Ser. No. 60/780,793, filed Mar. 8, 2006, and U.S. Ser. No. 60/780,749, filed Mar. 8, 2006, both of which are hereby expressly incorporated by reference in their entirety.

### BACKGROUND OF THE INVENTION

1. Field of the Invention

The present invention relates to methods of treating various neuroinflammatory diseases and conditions with retinoic acid and pharmaceutical preparations of retinoic acid useful in the treatment of neuroinflammatory diseases or conditions such as stroke, Alzheimer's disease, amyotrophic lateral sclerosis, multiple sclerosis, acute traumatic head injury, and the like.

2. Background of the Invention

Retinoids are a class of chemical compounds that are structurally related to vitamin A. They include the natural compounds and synthetic derivatives of retinol that often, but not always, exhibit vitamin A activity. Retinoids are used in medicine, primarily for their affects on the regulation of epithelial cell growth. Naturally occurring retinoid compounds such as all-trans-retinoic acid (tretinoin), 9-cis-retinoic acid, all-trans-3-4 didehydroretinoic acid, 4-oxo-retinoic acid, and 11-cis-retinol have pleiotropic effects that influence a large number of inflammatory, immune, and structural cells. Synthetically-derived retinoids such as 13-cis-retinoic acid (isotretinoin, trade-name Accutane®) etretinate, acitretin, and the polyaromatic retinoids or “arotinoid” compounds (e.g., Ro 13-7410 and Ro 15-1570) also possess pleiotropic activity.

Retinoids have been shown to modulate epithelial cell proliferation and differentiation through a series of nuclear receptors that belong to the steroid/thyroid receptor superfamily. The biological effects of retinoids have led to the development of many topical agents for dermatological disorders such as psoriasis, acne, and hypertrophic cutaneous scars, amongst others. Other medicinal applications of retinoids that have been suggested include the control of acute promyelocytic leukemia, adeno and squamous cell carcinoma, hepatic fibrosis, emphysema and other pulmonary disorders, angiogenesis, nephritis and wound healing. A general review of retinoids can be found in Goodman & Gilman's “The Pharmacological Basis of Therapeutics”, 10^{th} edition (2001, McGraw-Hill) Chapters 64-65, and U.S. Pat. Nos. 6,794,416, 6,787,131, 6,573,271, 6,372,753, 6,355,669, 6,339,107, 6,277,890, 6,248,749, 6,075,032, 6,071,924, 5,998,486, 5,973,007, 5,827,878, 5,821,254, 5,719,195, 4,985,235 and 4,129,662.

Microglia are central nervous system (CNS)-resident cells of hematopoietic origin with a phenotype resembling that of resident macrophages in other tissues. Although beneficial in various aspects of CNS maintenance and repair, microglia contribute to CNS damage by several pathways of neuroinflammatory or autoimmune pathology, including secretion of inflammatory cytokines (e.g., interleukin (IL)-6, tumor necrosis factor (TNF)-α, IL-1), secretion of nitric oxide, and, in the presence of T-cells, presentation of self and foreign antigens via phagocytic activity. Microglia also may contribute to the pathology of neurological disorders via non-inflammatory mechanisms, for instance via signaling pathways that accelerate neuronal apoptosis, or by altering amyloid beta metabolism. By these various mechanisms, microglia have been implicated in the progression or adverse outcomes of a number of diseases or injuries, including Alzheimer's disease (AD), multiple sclerosis (MS), amyotrophic lateral sclerosis (ALS), Parkinson's disease (PD), stroke, and variant Creutzfeldt-Jacob disease. Microglia may play a role in other neurological conditions including depression, chronic pain, and autism.

The discovery of an ever-greater role for activated microglia in neuroinflammatory diseases has led to their emergence as a target for therapeutic intervention, either to prevent or treat neuroinflammatory disease such as chronic neurodegenerative disorders, or to improve outcomes after acute episodes or injuries such as traumatic head injury. A known suppressor of microglial activation, minocycline, has been taken into clinical trials for PD and ALS based on pre-clinical data showing suppression of symptoms in animal models,

Although there are approved drugs currently prescribed for various neuroinflammatory disorders, these drugs often have limited efficacy and some have narrow therapeutic indexes. Thus, there is a significant need for new drugs to treat the various neuroinflammatory diseases.

### SUMMARY OF THE INVENTION

In accordance with the objects outlined herein, the present invention provides methods of treating a neuroinflammatory condition comprising administering to a patient in need thereof a therapeutically effective amount of a retinoid or a pharmaceutically acceptable salt, hydrate, solvate, or pro-drug thereof. These neuroinflammatory conditions include Alzheimer's disease (AD), multiple sclerosis (MS), amyotrophic lateral sclerosis (ALS), Parkinson's disease (PD), stroke, and variant Creutzfeldt-Jacob disease.

In an additional aspect, the invention provides methods of treating a patient to reduce or prevent microglial activation, and in particular microgial proliferation during neuroinflammation comprising administering to a patient in need thereof a therapeutically effective amount of a retinoid or a pharmaceutically acceptable salt, hydrate, solvate, or pro-drug thereof.

In a further aspect, the invention provides a variety of different retinoids that can be used in the methods of the invention, including, but not limited to, nonaromatic, monoaromatic and polyaromatic retinoids, including, but not limited to, retinol; retinal; retinoic acid (all-trans-retinoic acid, sometimes referred to as tretinoin); 9-cis-retinoic acid (sometimes referred to herein as alitretinoin) all-trans-3-4 didehydroretinoic acid, 4-oxo-retinoic acid; 11-cis-retinol; ethyl retinoate: 1-O-retinoyl-β-D-glucopyranuronic acid: retinal oxime; N6-retinylidene-L-lysine; deoxyretinol; neovitamin A; 4-Nitrobenzyl all-trans-retinoate; retro-retinoids including anhydro vitamin A, 4,14-retro-retinyl acetate and others disclosed in U.S. Pat. Nos. 5,358,972, 5,521,221, 5,648,563, 5,814,612, 5,908,868; Ro-13-6298; retinol, 13-cis-retinoic acid (isotretinoin, trade-name ACCUTANE®); etretinate; acitretin; arontinoid, adapalene; tazarotene; Ro-8757; compounds outlined in Benbrook, Mini Reviews in Medicinal Chemistry 2002 2:277 (incorporated by reference); hepaxanthin; ethyl 12-fluororetinoate; γ-vitamin A, nor retinoids including motretinide, and 5-acetyl4,18-dinor-retinoic acid;, arotinoids and heteroarotinoids (including Ro 13-7410 and Ro 15-1570).

In an additional aspect, the retinoids of the invention can be used in conjunction with additional therapeutic agents, particularly minocycline and glucoconticoids.

In a further aspect, the invention provides methods of treating multiple sclerosis, cerebro-vascular incidents including strokes, depression, human immunodeficiency virus-associated dementia, certain forms of chronic pain; autism, Huntington's disease, variant Cretzfeldt-Jakob disease; amylotrophic lateral sclerosis (ALS), multiple sclerosis, Alzheimer's disease, Parkinson's disease and traumatic brain injury.

### BRIEF DESCRIPTION OF THE DRAWINGS

FIG. 1: depicts that isotrentinoin Inhibits Microglial Proliferation in the LPS model of neuroinflammation. Female c57bl/6 mice were labeled with ²H₂O for 7 days and injected with LPS (1 mg/kg, i.p.) on days 1, 3 and 5. Accutane (40 mg/kg, i.p.) was administered daily for the duration of the study. Data represent mean±SD of 5 animals per group. * Indicates significant difference (p<0.0001) between Vehicle and LPS-treated groups, # indicates significant difference (p<0.0001) compared to LPS-treated animals. depicts inhibition of LPS-induced microglial proliferation. LPS (1 mg/kg) was given on days 1, 3, and 5 of treatment. Isotretinoin (40 mg/kg/day) and 8% ²H₂O label were given for 7 days commencing on Day 1.

FIG. 2 depicts the Dose Response of Accutane in the LPS model of neuroinflammation. Female c57bl/6 mice were labeled with ²H₂O for 7 days and injected with LPS (1 mg/kg, i.p.) on days 1, 3 and 5. Minocycline (45 mg/kg, i.p.) was dissolved in saline and injected twice a day for the first 3 days, and once a day for the rest of the study. Accutane (1, 4, 12 or 40 mg/kg, i.p.) was dissolved in DMSO and administered daily for the duration of the study. Data represent mean±SD of 5 animals per group. * Indicates significant difference (p<0.0001) between vehicle and drug-treated groups of animals injected with LPS.

FIG. 3 depicts the inhibition of Microglial Proliferation in the experimental autoimmune encephalomyelitis (EAE) model of neuroinflammation. Female c57bl/6 mice were labeled with ²H₂O for 7 days and received either sham or EAE immunizations. EAE was induced by subcutaneous injection of 100 μg synthetic MOG(35-55) peptide emulsified in 0.1 ml complete Freund's adjuvant on day 0. Pertussis toxin (400 ng) was injected intravenously on days 0 and 2. Minocycline (45 mg/kg, i.p.) was dissolved in saline and injected twice a day for the first 3 days, and once a day for the rest of the study. Accutane (30 mg/kg, i.p.) was dissolved in DMSO and injected once a day for the duration of the study. Data represent mean±SD of 5 animals per group. * Indicates significant difference (p<0.0001) between sham and EAE groups treated with vehicle, # indicates significant difference (p<0.0001) between vehicle and drug treated EAE groups.

FIG. 4 shows the effect of isotretinoin treatment in EAE induced by MOG peptide. Female C57BL/6 mice were immunized with MOG35-55 (EAE mice). All mice were labeled with 8% 2H2O for 1 week and received daily treatment with either vehicle or isotretinoin (10 mg/kg/day, ip). Clinical scores for EAE symptoms are shown. Treatment began the on the day before immunization. * Indicates p<0.05 compared to vehicle group for days 18-25 post-immunization. Data represent mean±SEM of 8 mice per group.

### DETAILED DESCRIPTION OF THE INVENTION

### I. OVERVIEW

The present invention is directed to methods and compositions for the treatment of neuroinflammatory diseases or disorders. In particular, the present invention provides for the use of retinoids or retinoic acids (RA) for the treatment of various diseases or disorders involving neuroinflammation.

Applicants have discovered that: 1) retinoic acids (RA), including the RA derivative isotretinoin (ACCUTANE®), potently suppress a cardinal feature of neuroinflammation, the proliferation of brain microglial cells under conditions of microglial stimulation, in addition to suppressing other components of the generalized neuroinflammatory response; 2) isotretinoin is as potent in this action as clinically established inhibitors of neuroinflammation, including minocycline and glucocorticoids; and 3) retinoids also reduce microglial proliferation and general neuroinflammation in art-accepted animal models of specific neuroinflammatory diseases, including the widely used model of multiple sclerosis (experimental autoimmune encephalomyelitis [EAE]).

### II. GENERAL TECHNIQUES

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987): PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999), all of which are incorporated by reference for the needed techniques. Furthermore, procedures employing commercially available assay kits and reagents will typically be used according to manufacturer-defined protocols unless otherwise noted.

### III. DEFINITIONS

By “stoxic effect” is meant an adverse response by a living system to a chemical entity or known drug agent. A toxic effect can be comprised of, for example, end-organ toxicity.

By “action” is meant a specific and direct consequence of an intervention such as the administering of a drug.

“Treatment” in this context includes delay in onset or severity of symptoms, retardation of mortality, and/or reduction of symptoms.

By “neuroinflammation” is meant the biological processes involved in the host inflammatory response within the brain in animals, including general components (influx of leukocytes, secretion of cytokines) and specific components (activation and proliferation of microglia).

By “microglia” is meant the resident monocytes (macrophages) in the brain, activation and proliferation of which is involved in the etiology and pathogenesis of many neuroinflammatory diseases or disorders. “Microglial activation” includes the recruitment of microglia to sites of inflammation as well as new microglia growth (e.g. increased microglia cell division).

By “neuroinflammatory diseases or disorders” is meant neurologic diseases or disorders characterized by the presence of inflammation in the brain. Examples of such disorders include multiple sclerosis, cerebro-vascular incidents including strokes, depression, human immunodeficiency virus-associated dementia, certain forms of chronic pain; autism, Huntington's disease, variant Cretzfeldt-Jakob disease; amylotrophic lateral sclerosis (ALS), multiple sclerosis, Alzheimer's disease, Parkinson's disease and traumatic brain injury, among many others well known in the art.

By “retinoids” or “retinoic acids” is meant the biochemical class of Vitamin A-related molecules that exhibit biological activity. Included within the definition of “retinoids” for the purposes of this invention include traditional retinoids as well as the arotinoids. The general structure of the parent compound of the retinoids and the arotinoids are shown as Structures 1 and 2:

Structure 1: parent compound of traditional retinoids:

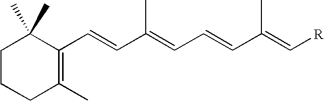

R in Structure 1 can be a number of different substituents, ranging from R═CH₂OH (Vitamin A), R═CHO (retinal), R═CO₂H (tretinoin or retinoic acid), R═CH₃ (deoxyretinol), R═CH₂OCOCH₃ (retinyl acetate), R═CH═NON (retinylamine), etc. In addition, R substitutents can be present on the cycloalkyl group or at any carbon. For example, a variety of substitutents can be used, such as alkyl substitutents (including heteroalkyl, cycloalkyl, heterocyclyaklyl, and substitutions thereof), aryl substitutents (including heteroaryl and substitutions thereof, two adjacent carbon atoms can be joined into cycloalkyl or aryl structures), etc. The methyl groups of Structure 1 can also be replaced by substitutent groups.

Structure 2: parent compound of arotinoids:

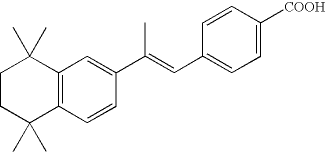

As above for Structure 1, any number of additional substituents, as well as the replacement of methyl groups, can be used.

Included within the definition of “retinoids” are both naturally occurring retinoids as well as derivatives thereof, including, but not limited to, nonaromatic, monoaromatic and polyaromatic retinoids, including, but not limited to, retinol; retinal; retinoic acid (all-trans-retinoic acid, sometimes referred to as tretinoin); 9-cis-retinoic acid, all-trans-3-4 didehydroretinoic acid, 4-oxo-retinoic acid; 11-cis-retinol; ethyl retinoate; 1-O-retinoyl-β-D-glucopyranuronic acid; retinal oxime; N6-retinylidene-L-lysine; deoxyretinol; neovitamin A; 4-Nitrobenzyl all-trans-retinoate; retro-retinoids including anhydro vitamin A, 4,14-retro-retinyl acetate and others disclosed in U.S. Pat. Nos. 5,358,972, 5,521,221, 5,648,563, 5,814,612, 5,908,868; Ro-13-6298 (see Stadler et al., Acta Derm. Venereol. 1984: 64(5):405, all of which are incorporated by reference); retinol, 13-cis-retinoic acid (isotretinoin, trade-name ACCUTANE®); etretinate; acitretin; arontinoid, adapalene; tazarotene; Ro-8757 (see Cariati et al., Oncogene 2003;22(6):906; compounds outlined in Benbrook, Mini Reviews in Medicinal Chemistry 2002 2:277 (incorporated by reference); hepaxanthin; ethyl 12-fluororetinoate; γ-vitamin A, nor retinoids including motretinide, and 5-acetyl-4,18-dinor-retinoic acid; arotinoids and heteroarotinoids (including Ro 13-7410 and Ro 15-1570, as well as the other Ro series of compounds). The definition includes both cis and trans derivatives as well as all stereoisomers. In addition, as will be appreciated by those in the art, the structures shown in Structures 1 and 2, as well as the compounds outlined herein, may have a variety of substitutents such as alkyl substitutents (including heteroalkyl, cycloalkyl, heterocyclyaklyl, and substitutions thereof), aryl substitutents (including heteroaryl and substitutions thereof), etc.

By “therapeutic action” or “therapeutic effect” is meant an effect on a biochemical or molecular process (i.e., the flow of molecules through metabolic pathways or networks) in a manner that is beneficial to the organism; e.g. any effect elicited by a compound or combination of compounds or mixtures of compounds that provides ameliorative or palliative results, or improves, even to the slightest degree, any clinical sign or symptom of a disease or condition. The effect may be responsible for, or contributing in, a causal manner to the initiation, progression, severity, pathology, aggressiveness, grade, activity, disability, mortality, morbidity, disease sub-classification or other underlying pathogenic or pathologic feature of one or more diseases wherein said effect is beneficial to health or otherwise contributes to a desirable outcome (e.g., a desirable clinical outcome).

By “therapeutically effective amount” is meant an amount effective to ameliorate the symptoms of, or ameliorate, treat or prevent neuroinflammation, including the activation (including proliferation) of migroglial cells.

By “condition” or “medical condition” is meant the physical status of the body as a whole or of one of its parts. The term is usually used to indicate a change from a previous physical or mental status, or an abnormality not recognized by medical authorities as a disease or disorder. Examples of “conditions” or “medical conditions” include, but are not limited to, obesity, cancer, proliferative diseases and pregnancy.

As used herein “pro-drug” refers to any compound which releases an active drug in vivo when such a compound is administered to a mammalian subject. Pro-drugs can be prepared, for example, by functional group modification of a parent drug. The functional group may be cleaved in vivo to release the active parent drug compound. Pro-drugs include, for example, compounds in which a group that may be cleaved in vivo is attached to a hydroxy, amino or carboxyl group in the active drug. Examples of pro-drugs include, but are not limited to esters (e.g., acetate, methyl, ethyl, formate, and benzoate derivatives), carbamates, amides and ethers. Methods for synthesizing such prodrugs are known to those of skill in the art.

In one embodiment, the invention provides methods of treating a neuroinflammatory disease or disorder comprising administering to a patient in need of treatment a pharmaceutical composition comprising a retinoid, including isotretinoin, or pharmaceutically acceptable salt, hydrate, solvate, or pro-drug thereof

### IV. FORMULATIONS

In therapeutic use for the treatment of neuroinflammatory conditions, diseases, or disorders, the compound(s) utilized in the pharmaceutical method of the invention are administered to patients diagnosed with one or more neuroinflammatory conditions, diseases, or disorders at dosage levels suitable to achieve therapeutic benefit. By “therapeutic benefit” is meant that the administration of compound(s) leads to a beneficial effect in the patient over time.

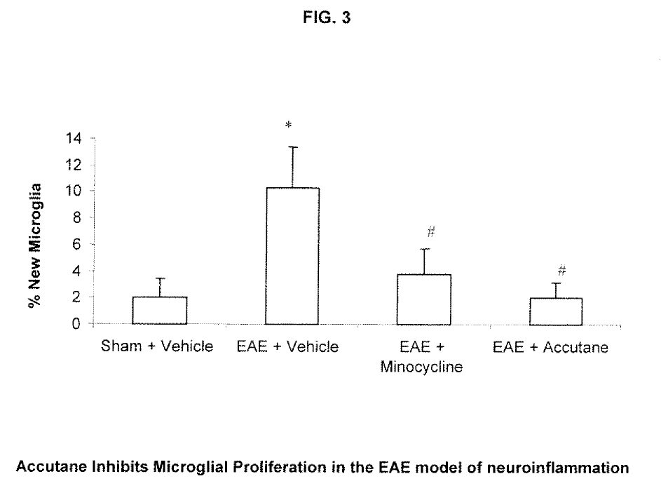

One retinoic acid may be administered alone or in combination with another retinoic acid. In addition, retinoic acids can be administered in combinations with other drugs/agents that inhibit microglial activation and/or proliferation. For example, one appropriate combination utilizes both a retinoic acid and minocycline. In fact, FIG. 3 shows for the first time that minocycline is effective in reducing microglial proliferation; thus the invention provides methods of reducing microglial proliferation comprising administering minocycline. In addition, other agents known to reduce microglial activation and/or proliferation can be used as well; for example, glucocorticoids and corticosteroids, including, but not limited to, naturally occurring and synthetic derivatives, including, but not limited to, hydrocortisone, aldosterone, cortisol, corticosterone, dexamethasone, glucocorticoid RU28362, etc.

For combinations, the compositions can be administered together in a single dosage form (e.g., oral formulations that combine the two drugs) or singly, in any of the dosage forms outlined below, simultaneously or sequentially. For example, one drug can be administered orally and the other intraperitoneally, either together or sequentially. In addition, when dosed separately, the dosages may be at different times or frequencies. Alternatively, the two drugs may be administered separately but in the same dosage form.

Initial dosages suitable for administration to humans may be determined from in vitro assays or animal models. For example, an initial dosage may be formulated to achieve a serum concentration that includes the IC₅₀ of the particular metabolically active agent of the compound(s) being administered, as measured in an in vitro assay. Alternatively, an initial dosage for humans may be based upon dosages found to be effective in animal models of neuroinflammation, such as the EAE mouse model. As one example, the initial dosage for each component of the pharmaceutical compositions outlined herein may be in the range of about 0.01 mg/kg/day to about 200 mg/kg/day, or about 0.1 mg/kg/day to about 100 mg/kg/day, or about 1 mg/kg/day to about 50 mg/kg/day, or about 10 mg/kg/day to about 50 mg/kg/day, can also be used. The dosages, however, may be varied depending upon the requirements of the patient, the severity of the condition being treated, and the compound(s) being employed. The size of the dose also will be determined by the existence, nature, and extent of any adverse side-effects that accompany the administration of a particular compound(s) in a particular patient. Determination of the proper dosage for a particular situation is within the skill of the practitioner. Generally, treatment is initiated with smaller dosages which are less than the optimum dose of the compound(s). Thereafter, the dosage is increased by small increments until the optimum effect under circumstances is reached. For convenience, the total daily dosage may be divided and administered in portions during the day, if desired.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Formulations suitable for oral administration can consist of (a) liquid solutions, such as an effective amount of the compound(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; (d) suitable emulsions and (e) aerosol formulations. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

The active compound or pharmaceutically acceptable salt thereof can be administered as a component of an elixir, suspension, syrup, wafer, chewing gum or the like. Syrup may contain, in addition to the active compounds, sucrose as a sweetening agent and certain preservatives, dyes and colorings and flavors.

As many of the retinoids described herein are acidic, they may be included in any of the above-described formulations as the free acid, a pharmaceutically acceptable salt, a pro-drug, solvate or hydrate. Pharmaceutically acceptable salts substantially retain the activity of the free acid and may be prepared by reaction with bases. Pharmaceutically acceptable salts include any known suitable salts of retinoids known in the art for administration to mammals. Pharmaceutical salts tend to be more soluble in aqueous and other protic solvents than is the corresponding free acid form. Similarly, the retinoids may be included in any of the above-described formulations as a solvate, hydrate or pro-drug. Preferred pro-drugs include hydrolyzable ester derivatives such as aromatic esters, benzyl esters and lower alkyl esters such as ethyl, cyclopentyl etc. Other pro-drugs are known to those of skill in the pharmaceutical arts.

The active compound or pharmaceutically acceptable salts thereof can also be mixed with other active materials that do not impair the desired action, or with materials that supplement the desired action.

As used herein, the term “pharmaceutically acceptable salt(s)” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counter-ion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The compound(s) of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like. In one embodiment, the pharmaceutically acceptable carrier is suitable for a electrohydrodynamic aerosol device, a nebulizer device or a aerosol device. In one preferred embodiment, the pharmaceutically acceptable carrier is a liquid such as water, alcohol, polyethylene glycol or perfluorocarbon.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound(s) of choice with a base, including, for example, liquid triglycerides, polyethylene glycols, and paraffin hydrocarbons.

Formulations suitable for parenteral administration, such as, for example, by intra-articular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, subcutaneous administration and intravenous administration are the preferred methods of administration. A specific example of a suitable solution formulation may comprise from about 0.1-100 mg/ml compound(s) and about 1000 mg/ml propylene glycol in water. Another specific example of a suitable solution formulation may comprise from about 0.1 or about 0.2 to about 100 mg/ml compound(s) and from about 800-1000 mg/ml polyethylene glycol 400 (PEG 400) in water.

A specific example of a suitable suspension formulation may include from about 0.2-30 mg/ml compound(s) and one or more excipients selected from the group consisting of: about 200 mg/ml ethanol, about 1000 mg/ml vegetable oil (e.g., corn oil), about 600-1000 mg/ml fruit juice (e.g., grape juice), about 400-800 mg/ml milk, about 0.1 mg/ml carboxymethylcellulose (or microcrystalline cellulose), about 0.5 mg/ml benzyl alcohol (or a combination of benzyl alcohol and benzalkonium chloride) and about 40-50 mM buffer, pH 7 (e.g., phosphate buffer, acetate buffer or citrate buffer or, alternatively 5% dextrose may be used in place of the buffer) in water.

A specific example of a suitable liposome suspension formulation may comprise from about 0.5-30 mg/ml compound(s), about 100-200 mg/ml lecithin (or other phospholipid or mixture of phospholipids) and optionally about 5 mg/ml cholesterol in water. For subcutaneous administration of a compound(s), a liposome suspension formulation including 5 mg/ml compound(s) in water with 100 mg/ml lecithin and 5 mg/ml compound(s) in water with 100 mg/ml lecithin and 5 mg/ml cholesterol provides good results.

The formulations of compound(s) can be presented in unit-dose or multi-dose sealed containers, such as ampoules and vials. Injection solutions and suspensions can be prepared from sterile powders, granules, and tablets of the kind previously described.

The pharmaceutical preparation is preferably in unit dosage form. In such form the preparation is subdivided into unit doses containing appropriate quantities of the compound(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents, discussed in more detail, below.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation (CA) and Gilford Pharmaceuticals (Baltimore, Md.). Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidylcholine, arachadoyl phosphatidylcholine, and cholesterol) in an inorganic solvent that is then evaporated leaving behind a thin film of dried lipid on the surface of the container. Aqueous solutions of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

### V. USES OF THE PRESENT INVENTION

The present invention provides pharmaceutical compositions for the treatment of neuroinflammatory conditions, diseases, or disorders. Examples of such conditions, diseases or disorders include, but are not limited to, stroke, multiple sclerosis, Amyotrophic Lateral Sclerosis (ALS), Alzheimer's Disease (AD), Huntington's Disease (HD), Parkinson's Disease (PD) Variant Cretzfeldt-Jakob Disease (vCJD), Human Immunodeficiency Virus (HIV)-associated dementia (HAD), Traumatic Brain Injury (TBI), Depression, chronic pain, and Autism. Table 1 depicts these conditions, diseases, or disorders and their associated animal models and clinical endpoints.

TABLE 1
Neuroinflammatory conditions, diseases, or disorders, animal
models, and clinical and neuroinflammatory endpoints

| Condition, Disease, or | | Clinical and |
|---|---|---|
| Disorder | Animal Model | Neuroinflammatory Endpoints |
| Stroke | Middle cerebral artery | Infarct size and neurological |
| | occlusion model for focal | scores are assessed in the ischemia |
| | ischemia; four-vessel | models. In focal ischemia, neuro- |
| | occlusion or bilateral | inflammation is characterized by an |
| | occlusion of the carotid | upregulation of CR3, MHC Class I and |
| | arteries for global ischemia | II, CD4 and CD8, as well as induction of |
| | | cytokines. Three principal patterns of |
| | | microglial activation can be |
| | | distinguished - rapid morphological |
| | | transformation into phagocytes in the |
| | | border zone of infarction, slowly |
| | | evolving response in degenerating |
| | | neuronal nuclei and fiber tracts, and |
| | | transient ipsilateral activation in |
| | | regions remote from the infarction. In |
| | | global cerebral ischemia, microglial |
| | | activation is most prominent and |
| | | persistent in areas of ongoing neuronal |
| | | death and axonal degeneration. |
| Multiple Sclerosis | Experimental Autoimmune | Clinical score for EAE is generated on |
| | Encephatomyelitis (EAE) | the basis of observed symptoms of |
| | model in mice and rats | paralysis. Neuro-inflammation is |
| | | characterized by infiltration of immune |
| | | cells from the periphery into the brain, |
| | | Recently, it has been shown by Heppner |
| | | et al (2005) that microglia play a |
| | | critical role in development and |
| | | maintenance of inflammatory lesions in |
| | | the CNS. Moreover, microglial activation |
| | | and proliferation has also been shown |
| | | to precede the onset of CNS autoimmunity |
| Amyotrophic Lateral | Transgenic mice and rats | Motor deficits in the animal models are |
| Sclerosis | expressing the SOD1 G93A | assessed using gait analysis and motor |
| | mutation | coordination and balance tests (rotarod, |
| | | paw grip endurance, loaded grid, beam |
| | | balance etc.). Neuroinflammation is |
| | | characterized by microgital and astrocytic |
| | | activation, upregulation of cell adhesion |
| | | molecules, increase in IgG and Fc receptor. |
| | | The inflammatory response correlated |
| | | with disease onset and progression |
| Alzheimer's Disease | APP (amyloid precursor | Behavior tests to assess learning and |
| | protein) transgenic mice, | memory deficits include the Morris water |
| | APP and Presenilin double | maze and active avoidance tests. |
| | transgenic mice, APP-PS-tau | Microglial activation and proliferation |
| | triple transgenic mice | has been consistently observed in AD |
| | | brain in regions with Aβ deposits along |
| | | with increased expression of cytokines. |
| | | Recently, microglial activation has been |
| | | shown to precede and accelerate amyloid |
| | | plaque deposition and also exacerbate |
| | | neuropathological features such as tangle |
| | | formation due to tau hyperphosphorylation. |
| Huntington's Disease | Quinolinic acid or 3- | Motor deficits are assessed by a battery |
| | nitropropionic acid induced | of behavioral tests to measure motor |
| | lesions in the rat | aspects of swimming, fore and hind limb |
| | Transgenic mouse models | coordination, balance and sensorimotor |
| | (R6/2 mice, N-171-82Q | gating. Immunohistochemical studies have |
| | mice) | found activated microglia in Huntington |
| | | disease brain, as well as in the |
| | | quinolinic acid model of HD. |
| Parkinson's Disease | MPTP (1-methyl-4-phenyl- | Motor deficits are assessed by behavioral |
| | 1,2,3,6-tetrahydropyridine) or | tests, such as the rotarod, swim test, |
| | 6-(OH)Dopamine induced | balance beam test, pole test, locomotor |
| | lesions in rats, mice or | activity tests, as well as gait analysis. |
| | monkeys | Microglial activation has been observed |
| | | in MPTP-treated mice and monkeys, as well |
| | | as in post-mortem PD brains. |
| Variant Cretzfeldt- | Scrapie or bovine spongiform | Mice develop progressive neurological |
| Jakob Disease | encephalopathy infections to | symptoms leading to death and |
| | mice or hamsters, transgenic | neuropathological changes include an |
| | mice expressing human | accumulation of mutant PrP in the brain. |
| | prion protein (PrP) | Microglial activation and increased release |
| | | of cytokines has been shown to occur in |
| | | prion encephalopathies. |
| Human Immunodeficiency | HIV-infected SCID mice, | Cognitive deficits are measured by |
| Virus- associated | High viral load simian | testing animals in the Morris Water Maze, |
| dementia | immunodeficiency virus (SIV) | Neurological dysfunction in HAD appears |
| | model | to be a consequence of microglial |
| | | activation leading to the production |
| | | and release of cytotoxic molecules. |
| Traumatic Brain Injury | Lateral fluid percussion injury | Cognitive deficits are assessed by |
| | model, cortical impact injury | testing animals in the Morris Water maze. |
| | model | Microglial activation and proliferation |
| | | has been reported in the brains of |
| | | animals and patients of TBI |
| Depression | Forced swim test (FST), tail | Behavioral tests assess ‘escape latency’ |
| | suspension test (TST), | in the FST, TST and LH tests, ‘anhedonia’ |
| | learned helplessness (LH), | in CMS is measured by decreased |
| | chronic mild stress (CMS), | preference for sucrose solution, locomotor |
| | olfactory bulbectomy, | hyperactivity or passive avoidance is |
| | sickness behavior | tested after olfactory bulbectomy. The |
| | | neuro-inflammatory component to depression |
| | | is thought to involve pro-inflammatory |
| | | cytokines (IL-1β, IL-2, IL-6, TNF-α) |
| | | and their interactions with the |
| | | neutransmitter and neuro-endocrine systems. |
| Chronic Pain | Sciatic chronic constrictive | Behavioral tests assess ‘hyperalgesia’ |
| | injury neuropathic pain | (heightened response to a noxious |
| | model, L5 spinal nerve | stimulus) or ‘allodynia’ |
| | injury-induced behavioral | (heightened response to a noxious stimulus). |
| | pain. | Neuroinflammation is characterized |
| | | by microglial and astrocytes activation |
| | | followed by production of cytokines, |
| | | cell adhesion molecules, chemokines and |
| | | expression of surface antigens. |
| Autism | None | Brain tissue from autism patients showed |
| | | microglial and astroglial activation, |
| | | increase in neuroglia-derived macrophage |
| | | chemoattractant protein (MCP)-1 and |
| | | tumor growth factor-β1. |

### I. EXAMPLES

The following non-limiting examples further illustrate the invention disclosed herein:

Mice: All animal studies were carried out according to NIH guidelines for the care and use of laboratory animals and received prior approval by the KineMed internal animal care and use committee. 9-10 week old female C57/Bl6 mice (Taconic, Oxnard, Calif.) were housed in a climate controlled environment with a 12 hour light/dark cycle and were fed standard rodent chow and water ad libitum. For heavy water labeling, animals received a priming IP bolus of 35 ml/kg 0.9% NaCl in 99.9% ²H₂O and were maintained on 8% ²H₂O in drinking water until sacrifice.

### Example 1

### Isotretinoin Reduces Microglia Proliferation in an Lipopolysaccharide (LPS)-Induced Mouse Model of Neuroinflammation

To induce neuroinflammation, C57/Bl6 mice received 1 mg/kg E. coli LPS (L4391, Sigma Chemical, St Louis, Mo.; serotype 0111:B4; stock of 0.2 mgs/ml in PBS) IP on days 1, 3, and 5 of ²H₂O labeling and were sacrificed the morning of day 8. In order to minimize dilution of body water ²H enrichment in animals due to the volume of injections, 5% ²H₂O solution was used as the vehicle for LPS. Isotretinoin (Sigma) was dissolved in DMSO and administered IP daily at 40 mg/kg, drug treatment was maintained throughout the labeling period.

Isolation and characterization of microglia: Anesthetized mice were transcardially perfused with ice-cold PBS (30 ml). Brains (without olfactory bulbs) were removed and kept in ice-cold PBS until processing. Tissue was passed once through a 1 mm mesh, and agitated in digestion buffer (12.5 ml of 150 mM NaCl, 5 mM KCl, 10 mM NaHCO3, 0.5 mM EDTA, 15 mM HEPES, 5 mM D-glucose, containing 0.5 mg/ml DNAse I [Roche] and 2.5 mg/ml trypsin [Sigma]) in a baffle flask for 25 minutes at 37° C. Trypsin was inactivated by the addition of 6.25 ml of media (1:1 Hams F10:DMEM, 10% FBS, 100,000 units/l penicillin G, 50 mg/l streptomycin sulfate, 2 mM L-glutamine). The mixture was then placed into a Seward model 80 stomacher (Thetford, Norfolk, UK) and processed on the lowest setting for 2 minutes (alternatively, the mixture can be triturated with a 10 ml serological pipette for 3 minutes), then filtered through a 120 μm mesh, diluted to 25 ml with media, and spun at 350×g for 10 minutes at 4° C. The pellet was suspended in 15 ml of 22% Percoll (Amersham Biosciences; 22% v/v in PBS containing with 10 mM D-glucose, 0.2% BSA, and 35 mM NaCl) overlayed with 2 mls of the same buffer without Percoll, and centrifuged for 30 minutes at 950×g. The pellet was washed twice in media and stained with anti-CD11b-PE and anti-F4/80-FITC (eBioscience, San Diego, Calif.). Small aliquots of cells from each animal were used for isotype and single stain controls. Cells were washed, fixed in 4% paraformaldehyde, stained with the membrane-permeant DNA dye, DRAQ5 (Alexis Biochemicals, San Diego, Calif.), and sorted on a Coulter EPICS Elite cell sorter. Microglia were isolated as CD11b+, F4/80+ cells after gating on DRAQ5+ events to exclude debris, and on forward scatter peak height versus area to exclude doublets. Alternatively, cells were stained with anti-CD45-PE, anti-F4/80FITC, and anti-CD11b-conjugated magnetic microbeads, and immediately isolated using a MACS MS column according to the manufacturer's instructions (conjugated microbeads and MACS columns from Miltenyi Biotec, Auburn, Calif.). Purity of magnetically isolated cells was subsequently confirmed by analysis on a Coulter XL cell analyzer.

Analysis of cell turnover: GC/MS analysis was used to measure ²H incorporation from ²H₂O into the deoxyribose moiety of purine deoxyribonucleotides during de novo DNA synthesis. Genomic DNA from microglia was isolated using a DNEasy tissue kit (Qiagen, Valencia, Calif.), and processed and analyzed by GC/MS. The fraction of newly divided microglia was calculated as the ratio of excess ²H enrichment in purine deoxyribose of microglial DNA to the corresponding enrichment in bone marrow DNA.

Statistical analysis: A t-test was used to compare two groups. For comparison of multiple groups, a one-way ANOVA test was used, with a post-hoc Tukey test to compare selected groups. Differences between groups were considered statistically significant at p<0.05.

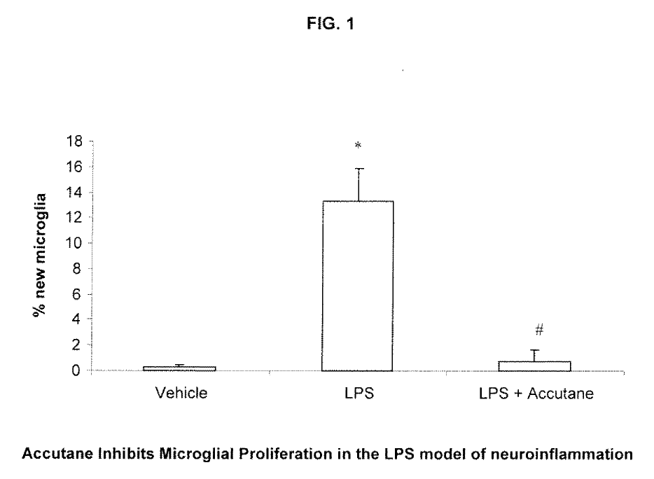

As can be seen in FIG. 1, isotretinoin dramatically inhibited LPS-induced microglial proliferation.

### Example 2

### Isotretinoin Reduces Microglia Proliferation in the Experimental Autoimmune Encephalitis (EAE) Mouse Model of Multiple Sclerosis

EAE was induced by subcutaneous injection of 100 μg synthetic MOG(35-55) peptide (QCB, Hopkinton, Mass.), emulsified in 0.1 ml complete Freund's adjuvant (CFA; incomplete Freund's adjuvant containing 4 mg/ml heat-killed M. tuberculosis H37Ra; both from Difco/Becton Dickinson), on day 0. Pertussis toxin (400 ng; List Biological) was injected intravenously on days 0 and 2. Sham-immunized animals were injected with CFA and PBS. Labeling with ²H₂O and treatment with isotretinoin (30 mg/kg/day) was initiated on the day of immunization, drug t.

Isolation and characterization of microglia, analysis of cell turnover, and statistical analysis were performed as in Example 1, supra.

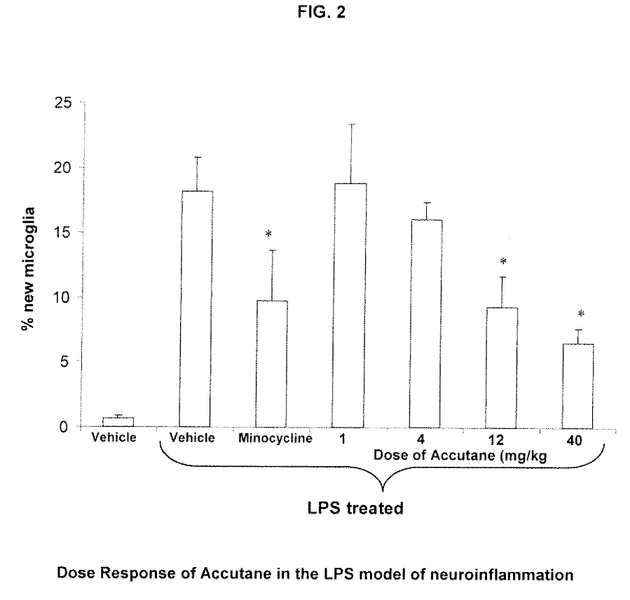

As can be seen in FIG. 2, isotretinoin dramatically inhibited microglial proliferation in the EAE model of multiple sclerosis.

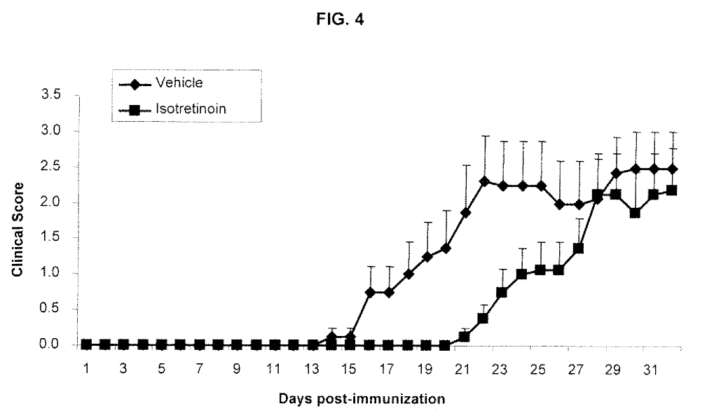

The next question addressed was whether inhibition of microglia proliferation was predictive of clinical response. Accordingly, the effect of isotretinoin was assessed on clinical symptoms of EAE during chronic treatment. Mice immunized with MOG₃₅₋₅₅ started developing clinical symptoms 14 days PI in the absence of treatment. Isotretinoin treatment delayed the onset of symptoms by 1 week (FIG. 4). Isotretinoin treatment also reduced the severity of clinical symptoms significantly (p<0.05) between day 18 and day 25 PI compared to vehicle-treated EAE mice. There were statistically significant effects of treatment (p<0.05) and days PI (p<0.001) upon clinical score, as well as a significant (p=0.006) interaction between these variables. The results show that isoretinoin inhibited microglia proliferation and delayed disease onset in EAE.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes to the same extent as if each individual publication, patent, or patent application were specifically and individually indicated to be so incorporated by reference.

1. A method of treating a neuroinflammatory condition comprising administering to a patient in need thereof a therapeutically effective amount of a retinoic acid or a pharmaceutically acceptable salt, hydrate, solvate, or pro-drug thereof.

2. A method according to claim 1, wherein said retinoic acid is isotretinoin.

3. A method according to claim 1, wherein said retinoic acid is a naturally occurring derivative.

4. A method according to claim 1, wherein said retinoic acid is a non-naturally occurring derivative.

5. A method according to claim 1, wherein said retinoic acid is selected from the group consisting of retinol, retinal, retinoic acid (all-trans-retinoic acid, sometimes referred to as tretinoin); 9-cis-retinoic acid, all-trans-3-4 didehydroretinoic acid, 4-oxo-retinoic acid; 11-cis-retinol; ethyl retinoate; 1-O-retinoyl-β-D-glucopyranuronic acid, retinal oxime; N6-retinylidene-L-lysine; deoxyretinol; neovitamin A; 4-Nitrobenzyl all-trans-retinoate; retro-retinoids including anhydro vitamin A and 4,14-retro-retinyl acetate, Ro-13-6298, 13-cis-retinoic acid (isotretinoin, trade-name ACCUTANE®); etretinate; acitretin; arontinoid, adapalene; tazarotene; Ro-8757, hepaxanthin; ethyl 12-fluororetinoate; γ-vitamin A, nor retinoids including motretinide, and 5-acetyl-4,18-dinor-retinoic acid, arotinoids and heteroarotinoids.

6. A method according to claim 1, wherein said method further comprises administering minocycline.

7. A method according to claim 1, wherein said method further comprises administering a glucocorticoid.

8. A method of treating a patient to reduce or prevent microglial activation comprising administering to a patient in need thereof a therapeutically effective amount of a retinoic acid or a pharmaceutically acceptable salt, hydrate, solvate, or pro-drug thereof.

9. A method according to claim 8, wherein said retinoic acid is isotretinoin.

10. A method according to claim 8, wherein said retinoic acid is a naturally occurring derivative.

11. A method according to claim 8, wherein said retinoic acid is a non-naturally occurring derivative.

12. A method according to claim 8, wherein said retinoic acid is selected from the group consisting of retinol, retinal, retinoic acid (all-trans-retinoic acid, sometimes referred to as tretinoin); 9-cis-retinoic acid, all-trans-3-4 didehydroretinoic acid, 4-oxo-retinoic acid; 11-cis-retinol; ethyl retinoate; 1-O-retinoyl-β-D-glucopyranuronic acid; retinal oxime; N6-retinylidene-L-lysine; deoxyretinol; neovitamin A; 4-Nitrobenzyl all-trans-retinoate; retro-retinoids including anhydro vitamin A and 4,14-retro-retinyl acetate, Ro-13-6298, 13-cis-retinoic acid (isotretinoin, trade-name ACCUTANE®); etretinate; acitretin; arontinoid, adapalene; tazarotene; Ro-8757, hepaxanthin; ethyl 12-fluororetinoate; γ-vitamin A, nor retinoids including motretinide, and 5-acetyl-4,18-dinor-retinoic acid, arotinoids and heteroarotinoids.

13. A method according to claim 8, wherein said method further comprises administering minocycline.

14. A method according to claim 8, wherein said method further comprises administering a glucocorticoid.
